(19) United States
(12) Patent Application Publication
(10) Pub. No.: US 2009/0110798 A1
(43) Pub. Date: Apr. 30, 2009

(54) PROCESS FOR OBTAINING PECTIN

(76) Inventors: Todd W. Gusek, Crystal, MN (US); Luca Zullo, Tonka Bay, MN (US); Aharon M. Eyal, Jerusalem (IL)

Correspondence Address: CARGILL, INCORPORATED, 15407 MCGINTY ROAD WEST, WAYZATA, MN, 55391, US

(21) Appl. No.: 11/908,272

(22) PCT Filed: Mar. 9, 2006
(86) PCT No.: PCT/US2006/008989

§ 371 Date: Jul. 29, 2008

Related U.S. Application Data
(60) Provisional application No. 60/659,936, filed on Mar. 9, 2005.

(51) Int. Cl. A23L 1/0524; C12P 19/04; C08B 37/06

(52) U.S. Cl. 426/577; 435/72; 536/2

(57) ABSTRACT

There is described a method for obtaining pectin from a pectin-containing material that involves treating the pectin-containing material with an enzyme that causes the pectin to be released from the pectin-containing material. Suitable enzymes include, but are not limited to, cellulase enzymes, hemicellulase enzymes, and mixtures thereof. Ethanol and isopropanol are typical alcohols utilized in recovering the pectin. Also decarbohydrates scribed is the pectin produced by the method, and the use of the pectin in foods and beverages.

### CROSS REFERENCE TO RELATED APPLICATIONS

This application claims priority to co-pending U.S. provisional patent application Ser. No. 60/659,936 filed Mar. 9, 2005, the entire contents of which are incorporated herein by reference.

### FIELD OF THE DISCLOSURE

The present disclosure relates to a process for obtaining pectin from a pectin-containing material, such as citrus fruit.

### BACKGROUND OF THE DISCLOSURE

A commercial process for producing pectin involves treating a citrus peel with an acid such as nitric or hydrochloric acid to achieve a pH of about 2 or below, and heating for a period of 1-3 hours. Following this treatment, the spent peel solids are separated from the pectin extract and isopropanol is added to the liquid fraction to precipitate the pectin. Pectin solids are filtered from the liquid supernatant. The pectin is then washed with more isopropanol prior to drying. The collected isopropanol is recovered by distillation and the spent peel can be dried and burned for fuel or pelletized for use in animal feed.

There is, however, a continuing need for a process that may produce enhanced yields of pectin from various pectin-containing materials.

### SUMMARY OF THE DISCLOSURE

The present disclosure relates to obtaining pectin from pectin-containing material. The process involves treating a pectin-containing material in an aqueous medium with an enzyme to release the pectin from the pectin-containing material. The resulting product is subjected to a separation method to separate insoluble residue from the pectin solution. The released pectin may then be suitably recovered by any conventional technique, such as by contacting the pectin solution with an alcohol, such as, for example, isopropanol or ethanol. Also, the disclosure relates to use of the pectin in foods and beverages.

### DETAILED DESCRIPTION OF THE DISCLOSURE

The present disclosure relates to obtaining pectin from pectin-containing material. The process involves treating a pectin-containing material in an aqueous medium with an enzyme to release the pectin from the pectin-containing material. The resulting product is subjected to a separation method to separate insoluble residue from the pectin solution. The released pectin may then be suitably recovered by any conventional technique, such as by contacting the pectin solution with an alcohol such as, for example, isopropanol or ethanol.

The term pectin-containing material, as used in the present process means any source of pectin that may be enzymatically treated to release the pectin. Exemplary of pectin-containing materials include, but are not limited to, any citrus fruit such as limes, lemons, oranges, grapefruits and tangerines, or the like; also suitable for use are tropical fruits such as guava, papaya, passion fruit, mango and the like; and other sources that are suitable such as apples, sugar beets, sunflowers, soybeans and the like. Mixtures of the pectin-containing materials may be used. In one embodiment, the pectin-containing material is a citrus fruit or an apple. In another embodiment, the pectin-containing material is the peel of a fruit, particularly a citrus fruit, wherein at least a portion of the liquid has been removed from the fruit. In another embodiment, the pectin-containing material is ground to a smaller size prior to or during treatment with an enzyme in the present process.

The aqueous medium used in the process to allow the enzyme treatment to occur may be any aqueous medium. In one embodiment, the aqueous medium may be water. In another embodiment, the aqueous medium may be acidified water. In another embodiment, the aqueous medium may be water that contains organic and inorganic salts, chelating agents, ions, oxidizing agents, reducing agents and the like. In yet another embodiment, the aqueous medium may be a recycled aqueous medium, for example, the aqueous medium resulting from pectin recovery and alcohol distillation. Without intending to be limiting, the aqueous medium used in the process may be a combination of one or more of the aforementioned aqueous media, or other aqueous media. The amount of aqueous medium to be utilized is any amount that will allow the treatment of the pectin-containing material with the enzyme to occur, to thereby release the pectin from the pectin-containing material.

In the present process, there may be utilized any enzyme, or mixture of enzymes, in the treatment of a pectin-containing material to cause the pectin to be released. Any amount of enzyme may be used, provided the enzyme will cause the pectin to be released. In one embodiment, 20 IU (international units) per g. dry peel solids to 210 IU per g. dry peel solids can be used, where one IU liberates one micromole of reducing sugar (expressed as glucose equivalent) in one minute under assay conditions of pH 4.8 and 50° C. Exemplary of suitable enzymes are cellulase and hemicellulase enzymes, individually or in combination. In one embodiment, examples of cellulases that are suitable for use in the present process include, but are not limited to, endo-glucanases, exo-glucanases, cellobiohydrolases, and the like, and mixtures thereof. Exemplary of hemicellulases that are suitable enzymes for use in the present process to release pectin from the pectin-containing material include, but are not limited to, xyloglucosidases, xylosidases, fucosidases, galactosidases, endoglucanases, mannosidases, glucuronidases, feruloyl esterases, endoxylanases, acetyl xylan esterases, xylanases, arabinofuranosidases, and the like, and mixtures thereof. Among the many enzymes suitable for use herein, the following are exemplary:

Available from AB Enzymes GmbH, of Feldbergstrasse 78, D-64293, Darmstadt, Germany, are Ecostone, Biotouch, Rohament, Veron CP, Ecopulp, Econase, and Veron 191 enzymes;

Available from Genencor International, of 925 Page Mill Road, Palo Alto, Calif. 94304-1013 are Multifect A40, Multifect xylanase, Optimash BG, Spezyme CP, and GC 220 enzymes;

Available from Novozymes A/S of Krogshoejvej 36 2880 Bagsvaerd, Denmark, are Cellusoft, Celluzyme, Cellulast, Fungamyl, Viscozyme, Alcalase 2.4 L FG, Novozym FM 2.0 L enzymes, and the like;

Available from Dyadic International, of 140 Intracoastal Pointe Drive, Suite 404, Jupiter, Fla., 33477-5094, are Rocksoft, Cellustar, Viscostar, Fibrezyme, Brewzyme, acid cellulase no. 1, acid cellulase no. 2, neutral cellulase no. 1, neutral cellulase no. 2, and beta-glucanase BPC enzymes, and the like;

Available from Valley Research of 3502 North Olive Road, South Bend, Ind. 46628, are Cellulase 4000, Validose enzymes, and the like;

Available from Deerland Enzymes, of 1680 Roberts Blvd., Suite 406, Kennesaw, Ga. 30144, are Cellulase TR, Pentosanase enzymes, and the like;

Available fiom Lyven, of Zac Normandial-11, Avenue du pays de Caen, 14460, Columbelles, France are Cellulyve enzymes, and the like;

Available from Enmex, of Rio Lerma No. 228 Fracc., Ind. San Nicolas Tlalnepantla, Edo. De Mexico 54030 Mexico, are Stonezyme enzymes, and the like;

Available from Maps (India) Limited, of 302, Shapath-3, Near GNFC Info Tower, S.G. Road, Ahmedabad, 380054, India, are Palkofeel, Palkosoft, Palkobake enzymes, and the like;

Available from Bio Sun are Biocitruzyme, Cellulase FG enzymes, and the like;

Available from Biocatalyst are Depol 740 L, Cellulase 13 L-CO12 L, Depol 692 L enzymes, and the like;

Available from Lucigen Corporation, 2120 West Greenview Drive Suite 9, Middleton, Wis. 53582 are Comblaster HE-1 and Comblaster HE-3.

As mentioned herein any of the enzymes suitable for use herein to release pectin from the pectin-containing material, may be used individually, or in combination.

In the present process, a sufficient amount of an aqueous medium is present to allow the enzymatic treatment of the pectin-containing material to occur, thereby to release the pectin from pectin-containing material and thereby form a pectin solution. Typically, the aqueous medium is present in a weight ratio of about 300 parts of aqueous medium to about 1 part of pectin that is present in the pectin-containing material. In one embodiment, the amount of aqueous medium ranges from about 150 parts of aqueous medium to about 1 part of pectin in the pectin-containing material. In another embodiment, the amount of aqueous medium utilized ranges from about 75 parts of aqueous medium to about 1 part of pectin in the pectin-containing material. In a still further embodiment, the amount of aqueous medium to be utilized in the process ranges from about 50 parts of aqueous medium to about 1 part of pectin in the pectin-containing material.

In the present process, any amount of aqueous medium can be present with the pectin-containing material to achieve release of the pectin and thereby form a pectin solution. In one embodiment, the aqueous medium is added to the pectin-containing material. Without intending to be limiting, any amount of aqueous medium may be added to the pectin-containing material prior to or during enzyme treatment, to assist in recovery and processing of the released pectin, for example, to adjust the viscosity of the resulting pectin solution.

As indicated herein, if desired, the pectin-containing material may be ground by any conventional method to reduce the size of the pectin-containing material, prior to or during the enzyme treatment. It may also be advantageous to grind the pectin-containing material prior to, or after the addition of, an aqueous medium to the pectin-containing material.

In one embodiment, the enzymatic treatment of the pectin-containing material is carried out under acidic conditions. In another embodiment, the acidic condition of the process is maintained at a pH of lower than about 7; in another embodiment at a pH of lower than about 5; and in still a further embodiment at a pH of lower than about 4. Typically, the pH will not be lower than about 2.

In one embodiment, the enzymatic treatment of the pectin-containing material is carried out herein at any temperature that results in the pectin being released from the pectin-containing material. In one embodiment, the enzymatic treatment is achieved at a temperature lower than about 100° C.; in another embodiment, at a temperature lower than about 80° C.; and in a further embodiment, at a temperature of lower than about 60° C. Typically, the temperature will not be lower than about 0° C.

In one embodiment, the enzymatic treatment of the pectin-containing material is carried out for any period of time that is sufficient to allow the pectin to be released from the pectin-containing material. In one embodiment, the enzymatic treatment of the pectin-containing material is carried out for a period of time less than 72 hours; in another embodiment, for a period of less than 24 hours; in another embodiment, for a period of less than about 12 hours; and in a further embodiment, for a period of less than about 6 hours. Typically, the period of time will not be less than about 1 hour.

The enzymatic treatment of the pectin-containing material yields a product that is, in general, comprised of insoluble residue and a pectin solution comprising released pectin in an aqueous medium. The resultant product may then be subjected to any conventional technique to separate the pectin solution from the insoluble residue. Typical methods for separating the pectin solution from the insoluble residue are filtration, centrifugation, and the like.

Subsequently the pectin may be recovered by contacting the pectin solution of the process with an alcohol such as ethanol or isopropanol.

In another embodiment, the pectin-containing material may be pretreated to stabilize the pectin-containing material. In such an embodiment, the pectin-containing material is treated to inactivate enzymes, such as pectin methylesterases, to stabilize the pectin-containing material. In one embodiment, the pectin-containing material is a citrus fruit or apple from which at least a portion of the liquid of the fruit has been removed. In another embodiment, the pectin-containing material is a peel. Pretreatment of the peel stabilizes the peel against deterioration by the enzymes present in the peel. The pretreatment occurs prior to the enzymatic treatment of the pectin-containing material to release the pectin.

Inactivation of the enzymes in the pectin-containing material may be achieved using any known technique. For example, in one embodiment, the pectin-containing material may be blanched with hot water, at a temperature of about 80 to about 100° C. for about 3 to 5 minutes. In another embodiment, the enzyme inactivation pretreatment may be achieved by heating using any conventional means, such as a modified jet cooker, extrusion, pressurized steam or steam explosion, application of radio frequency, microwave energy, acoustic energy such as ultrasound, or high pressure.

Pectin produced herein may be used in producing foods and beverages.

### OTHER EMBODIMENTS OF THE DISCLOSURE

The following is related to other embodiments of the disclosure. In particular, another embodiment relates to a method for processing citrus fruit that provides for producing ethanol from the non-juice part of the citrus fruit. The resultant ethanol could then be used in the process for obtaining pectin described herein, and provide an economic advantage. A description of several of the embodiments are as follows:

(A) A method for processing citrus fruit comprising
a. extracting juice from the fruit to form a juice product and a residue
b. selectively converting a residue comprising a cellulosic material and a pectinic material to products, which conversion is characterized in:
   i. converting at least about 20% of the cellulosic material to a water soluble carbohydrate and/or a product thereof (e.g. for fermentation without substantial destruction of pectin), and
   ii. that at least about 10% of the pectinic material is kept at a molecular weight of at least 10,000 Daltons (alternatively: kept in a form having less than about 5% solubility in an aqueous-ethanol solution comprising at least 10% ethanol).
      The method of A, wherein the citrus fruit is selected from a group consisting of oranges, grapefruit, lemons, limes, tangerines and the like.
      The method of A, wherein extracting comprises the use of equipment such as squeezer type extractors (for example FMC) and reamer-type extractors (for example Brown) and the like.
      The method of A, wherein the residue comprises at least one of the peel, rag, core, pulp, membranes, frits and the like of the citrus fruit.
      The method of A, wherein the residue is further selected from a group consisting of wet washed pulp or core resulting from a pulp washing or core washing system, and frits, peel particles and sludge resulting from an oil recovery system.
      (B) The method of A, wherein converting of cellulosic material comprises hydrolysis.
      The method of B, wherein hydrolysis forms water-soluble sugars such as glucose, cellobiose, arabinose, xylose, etc. (e.g. from hydrolysis of hemicellulose).
      (C) The method of B, wherein the hydrolysis or conversion is facilitated by a chemical catalyst, such as an acidic compound and/or by a biological catalyst, such as an organism and/or enzyme (or mixture of different enzymes) with cellulase activity.
      The method of C, wherein the catalyst is biological.
      (D) The method of A, wherein converting of cellulosic material comprises fermenting at least one cellulose hydrolyzate to at least one fermentation product.
      (E) The method of A, wherein the residue component also comprises at least one water-soluble carbohydrate, further comprising a step of fermenting the water-soluble carbohydrate to at least one fermentation product.
      (F) The method of D and E, wherein fermenting of at least one cellulose hydrolyzate and fermenting at least one water-soluble carbohydrate are conducted simultaneously, preferably in the same vessel.
      The method of D, E, and F, wherein the fermentation product is selected from a group consisting of ethanol, organic acids, amino acids, salts of any of those acids, proteins, carotenoids, enzymes and single-cell protein.
      (G) The method of A, further comprising a step of separating pectinic material from a residue component.
      The method of G, wherein separating comprises contacting with an acidic material or is biologically catalyzed with a suitable enzyme or organism.
      The method of G, wherein separating forms an aqueous medium containing the pectinic material and less than about 30% of the cellulosic material.
      (H) The method of A, wherein a residue component comprises at least one water-soluble carbohydrate, further comprising a step of separating the at least one water-soluble carbohydrate.
      The method of H, wherein separating comprises at least one of pressing and contacting with water or with an aqueous solution such as lime or acid solution.
      (I) The method of A, wherein a residue component comprises a fatty material such as D-limonene and peel oil, further comprising a step of separating the fatty material.
      The method of I, wherein separating comprises at least one of distillation, solvent extraction, de-emulsification and enzyme treatment.
      (J) The method of A, wherein a residue component comprises at least one phenolic compound, further comprising a step of separating the at least one phenolic compound.
      The method of J, wherein the phenolic compound is hesperidin and/or another flavonoid.
      (K) The method of A, wherein a residue component comprises at least one of carotenoid, pigment, essence, flavor component and limonoid glucoside, farther comprising a step of separating the at least one of carotenoid, pigment, essence, flavor component and limonoid glucoside.
      The method of K, wherein separating comprises at least one of extraction (e.g. using d-limonene, ethanol and/or their mixture as an extractant), distillation, precipitation, solvent-aided precipitation, chromatographic separation and membrane separation.
      The method of A, wherein a water-insoluble material is left after step (b), further comprising a step of separating such water insoluble material.
      (L) The method of A, comprising steps of separating from a residue material at least one of pectinic material, a water-soluble carbohydrate, D-limonene, a phenolic compound, carotenoid, pigment, essence, flavor component and limonoid glucoside and wherein the separating is conducted prior to step (b), simultaneously with it, after it or after separating water insoluble material.
      (M) The method of L, comprising fermenting to at least one product, wherein the separating and fermenting are conducted at any order or simultaneously.
      (N) The method of L OR M, comprising steps of separating from a residue material at least two of pectinic material, a water-soluble carbohydrate, D-limonene, a phenolic compound, carotenoid, pigment, essence, flavor component and limonoid glucoside and wherein separating at least one of those, separating at least another one of those, step (b), separating residual water insoluble material and/or fermentation are conducted at any order or simultaneously.
      The method of L, M and N, wherein separating of soluble materials is conduced after step (b) and after separating of residual water insoluble material.
      (O) The method of A, further comprising a step of precipitating a pectinic material from an aqueous medium.
      (P) The method of 0, wherein the aqueous medium comprises at least one of ethanol, an aluminum salt, an iron salt and a calcium ion.
      The method of P, wherein the aqueous medium is formed by fermenting carbohydrates in an aqueous medium comprising a pectinic material using for that purpose an organism with no pectinase activity or with low pectinase activity.
      The method of P, wherein the aqueous medium is formed by adding ethanol to an aqueous medium comprising a pectinic material.
      (Q) The method of A, wherein the fermentation product is ethanol and wherein the ethanol is separated from process streams and concentrated by distillation.
      The method of Q, wherein the concentrated ethanol is dried, e.g. by a method such as contact with molecular sieve.
      (R) A method for the manufacture of ethanol and pectin comprising the steps of:
providing citrus peel material comprising pectin and carbohydrates; fermenting carbohydrates of citrus peel material to form at least one fermentation product;
generating an aqueous medium comprising pectin and ethanol; and
separating ethanol and pectin from the aqueous medium (e.g. and using the separated ethanol to assist in the recovery of the pectin).
The method of R, further comprising fermenting sugar in situ (e.g. fermenting sugars in the presence of pectin).
   (S) The method of R, further comprising a step of forming an aqueous solution of carbohydrates.
   The method of S, wherein forming the aqueous solution involves at least one of separating water-soluble carbohydrates and hydrolyzing a cellulosic material.
   (T) The previous methods, further comprising a step of treating a residue component or a citrus peel material prior to converting, extracting of soluble carbohydrates and/or hydrolyzing water-insoluble carbohydrates.
   The method of T, wherein treating involves at least one of grinding, treatment with a lime solution, or an acidic solution, distillation or steam distillation of volatile components such as peel oil and separating of at least one of pectin, D-limonene, hesperidin (e.g. for the production of high intensity sweetener), carotenoid, pigment, essence, limonoid glucoside and flavor component.
   The method of R, wherein fermenting is of an aqueous solution comprising carbohydrates and pectin.
   The method of R, wherein the at least one fermentation product is ethanol.
   (U) The previous methods, wherein a non-fermented component of the citrus peel material is used for energy.
   (V) The previous methods, wherein a non-fermented component of the citrus peel material is used as an ingredient in animal feed, e.g. combined (and dried) with citrus pulp pellets (CPP).
   (W) The previous methods, wherein a non-fermented component and a fermentation product of the citrus peel material are used as ingredients in animal feed, e.g. combined (and dried) with CPP.
   (X) The previous methods, wherein at least one carbohydrate resulting from the residue material (e.g. separated or formed on hydrolysis) is separated and used for commercial applications, e.g. as a sweetener.
   The method of X, wherein separating uses at least one of chromatographic method and precipitation of non-carbohydrate materials.
   (Y) A method comprising the steps of:
providing citrus peel material;
extracting water-soluble carbohydrates from the citrus peel material to form an aqueous solution of water-soluble carbohydrates and a fibrous residue and separating between the solution and the residue;
solubilizing pectin in the fibrous material to form an aqueous medium comprising pectin and a second residue and separating the aqueous medium from the second residue;
hydrolyzing water-insoluble carbohydrates in the second residue to form a solution of hydrolyzate;
fermenting carbohydrates in at least one of the aqueous solution of step (b) and the hydrolyzate of step (d) to form ethanol;
using the ethanol to precipitate pectin from the pectin-containing aqueous medium of step (c) and
distilling ethanol.
   (Z) A method comprising the steps of:
a. providing citrus peel material;
b. hydrolyzing water-insoluble carbohydrates in the citrus peel material to form an aqueous solution comprising carbohydrates and pectin
c. fermenting carbohydrates in the aqueous solution to ethanol
d. precipitating pectin (e.g. with ethanol) from the aqueous solution and
e. distilling ethanol.
   (AA) The previous methods wherein a solution comprising galacturonic acid and/or its salt is formed, further comprising the step of converting the acid or the salt to ascorbic acid.
   (BB) A carbohydrates preparation containing at least one of sucrose, fructose and glucose produced according to any of the above methods.
   (CC) A pigment and/or flavoring and/or essence product produced according to any of the above methods. The pigment is comprised of carotenoid compounds and the like. Flavors and essences are comprised of alcohols, aldehydes, esters, hydrocarbons and like compounds.
   (DD) A dietary fiber produced from a non-fermented component produced according to any of the above methods. The dietary fiber is comprised of complex carbohydrates including both water soluble and water insoluble pectins, cellulose and hemicellulose. The dietary fiber may also comprise protein.

### BRIEF DESCRIPTION OF THE DRAWINGS

FIG. 1 is a flow diagram of a method for processing citrus fruit according to an exemplary embodiment.

FIG. 2 is a flow diagram of a method for processing citrus fruit according to an alternative embodiment of the present disclosure.

### DETAILED DESCRIPTION OF ALTERNATIVE EMBODIMENTS OF THE DISCLOSURE

According to a method for processing citrus fruit to separate pectin and produce an alcohol (e.g. ethanol), juice is extracted from a citrus fruit, leaving a residue comprising non-juice components of the fruit (referred to in the following as “residue”). Any citrus fruit is suitable, e.g. oranges, grapefruit, lemons, limes, tangerines and the like. Juice extraction may use equipment well known to the juice industry, such as squeezer type extractors (for example FMC) and reamer-type extractors (for example Brown). According to another embodiment, all the residue is treated in the process and no separation of residues components takes place before the process. According to an alternative embodiment, selected fractions of the residue are treated separately, e.g. when recovery of a component is desired and where that component is concentrated in selected fractions. The treated material is referred to in the following as “residue component”. According to another embodiment, the residue is comminuted, e.g. ground prior to further treatment.

According to an exemplary embodiment, the residue component comprises a cellulosic material and a hemicellulosic material and a pectinic (or “pectinaceous”) material and is selectively converted to products. According to an embodiment, a significant fraction of the cellulosic material is converted to water-soluble carbohydrates and/or at least one product thereof. That fraction is at least about 20%, suitably at least about 40%, more suitably at least about 60%. According to one embodiment, a significant fraction of the pectinic material is not hydrolyzed, or otherwise converted to products of molecular weight lower than about 10,000 Daltons. Alternatively, a significant fraction of the pectinic material is kept in a form having less than about 5% solubility in an aqueous-ethanol solution comprising at least 10% ethanol.

According to another embodiment, the cellulosic material of the residue component is hydrolyzed to glucose. Any cellulose selective catalyst could be used to facilitate the hydrolysis, e.g. a chemical catalyst (e.g. an acidic material) or a biological catalyst (an enzyme and/or an organism with cellulase activity). The term “selective” as used in this disclosure refers to the ability of the catalyst to hydrolyze cellulosic material. The catalyst is selected so that it efficiently hydrolyzes cellulosic material. Particularly attractive are biological catalysts of high cellulase activity. Endogenous enzymes could also be used.

According to an alternative embodiment, the cellulosic material of the residue component is converted to at least one fermentation product, e.g. by organisms capable of hydrolyzing it and fermenting the hydrolyzate. Fermentation could (also) be of hydrolyzate formed by another method (e.g. using chemically or enzymatically catalyzed hydrolysis) and/or water-soluble carbohydrates present in the residue material.

One or more fermentation products could be formed on using a suitable fermenting organism and fermentation conditions. Such products may include ethanol (e.g. for beverages and/or fuel), citric acid (for food and industrial application), acetic acid (e.g. for food and de-icing), various other carboxylic acids, amino acids, salts of any of citric, acetic, carboxylic and amino acids, proteins and single-cell proteins (e.g. for feed applications), carotenoids and enzymes (e.g. cellulase). Preferred products are selected based on commercial requirements. According to another embodiment, at least a fraction of the carbohydrates are fermented to an alcohol (e.g. ethanol), acetic acid and/or citric acid, or an amino acid.

According to another embodiment, water-soluble carbohydrates are separated from the residue material prior to the step of converting or simultaneously with it. Such separation may use at least one method such as enzymatic treatment, pressing and extraction with water, with an aqueous solution or with a lime solution or with an acidic solution. According to another embodiment, separated carbohydrates are combined with products of cellulose hydrolysis prior to fermentation. According to an alternative embodiment, those are fermented separately.

According to an alternative embodiment, pectinic material is separated from a residue component, e.g. by contacting with an acidic material or with a suitable enzyme, and forms an aqueous medium containing the pectinic material and less than about 30% of the cellulosic material. In case separating uses a contact with an acid, the conditions according to another embodiment are pH in the range of between about 1 and about 2.5, a temperature of between about 50° C. and about 130° C. and residence time of between about 1 and 200 minutes. While such separation could be done at any stage, conducting it prior to converting the cellulosic material enables the use of most efficient means for converting cellulosic material, without converting more pectinic material to lower molecular weight forms than desired.

The residue material typically comprises a number of components of relatively high commercial value. Those include D-limonene (e.g. to be used as a solvent or biocide); phenolic compounds of nutraceutical value and/or ones that provide precursors to other attractive products (e.g. hesperidin); carotenoids, pigments, flavor components, essences and limonoid glucosides. According to another embodiment, those components or some of them are separated from the residue material at any suitable stage of the process (e.g. prior to conversion of cellulosic material, after it or simultaneously with it). Separation of such compounds may use known methods, such as enzymatic treatment, extraction, crystallization, distillation, precipitation, adsorption, membrane separation, ion-exchange, solvent extraction (e.g. with D-limonene, ethanol and/or their mixture), etc.

According to another embodiment, pectin is produced from the residue component. Pectin is dissolved in water, aqueous solution or in one of the process streams, e.g. by the action of an acid or a suitable enzyme, to form an aqueous medium containing it. While various methods can be used to separate it, precipitation is preferred. According to another embodiment, precipitation is facilitated by the existence of solvent in the solution, e.g. an alkanol, e.g. ethanol or iso-propanol or acetone. Ethanol could be formed in the solution by fermenting carbohydrate or added from another source. According to an alternative embodiment, pectin is separated by the addition of at least one of aluminum salt, iron salt, calcium salt and calcium base. Optionally, the pectin-comprising solution is concentrated prior to the precipitation, e.g. using methods such as microfiltration, ultrafiltration, reverse osmosis and water evaporation. The separated pectin could be further purified, e.g. by washing with water-alkanol solutions, and dried, e.g. with hot air.

Ethanol formed by fermentation is separated from the fermentation liquor and from other streams, such as the ethanol containing solution left after pectin precipitation. Separation and concentration use distillation according to. another embodiment. The separated and concentrated ethanol could be dried, e.g. by contact with molecular sieves. The non-volatile components of the fermentation liquor present an attractive feed ingredient and could be used to increase the feed quality of other feed components, including insoluble fractions of the process and CPP.

According to another embodiment of the method for processing a citrus fruit, ethanol and pectin are produced from the residue material (also referred to here as citrus peel material). According to another embodiment, juice is extracted from the fruit and separated from the residual citrus peel material, which comprises pectin and carbohydrates. Those carbohydrates are fermented (as such or after further treatment, such as hydrolysis) to form at least one fermentation product. According to another embodiment the process also comprises generating an aqueous medium comprising pectin and ethanol and separating ethanol and pectin from the aqueous medium.

According to an embodiment of the method, a non-fermented material is used for energy, as an ingredient in feed or as a dietary fiber. Such material may include non-hydrated fiber and residues of ethanol distillation. In case of use in feed, other components could be added, e.g. fermentation products.

According to an embodiment of the process, carbohydrates separated from the residue and/or formed on hydrolysis are separated and used as sweeteners. Separation methods may include chromatographic separation and/or precipitation of insoluble materials including non-carbohydrate materials.

According to an embodiment of the method, the residue material or the citrus peel material is treated, prior to converting or simultaneously with it at least one method of grinding, heat treatment, extrusion, treatment with a lime solution, an acidic solution, enzymatic treatment, distillation or steam distillation of volatile components such as peel oil and separating of at least one of pectin, D-limonene, hesperidin, carotenoid, pigment, essence, limonoid glucosides and flavor component.

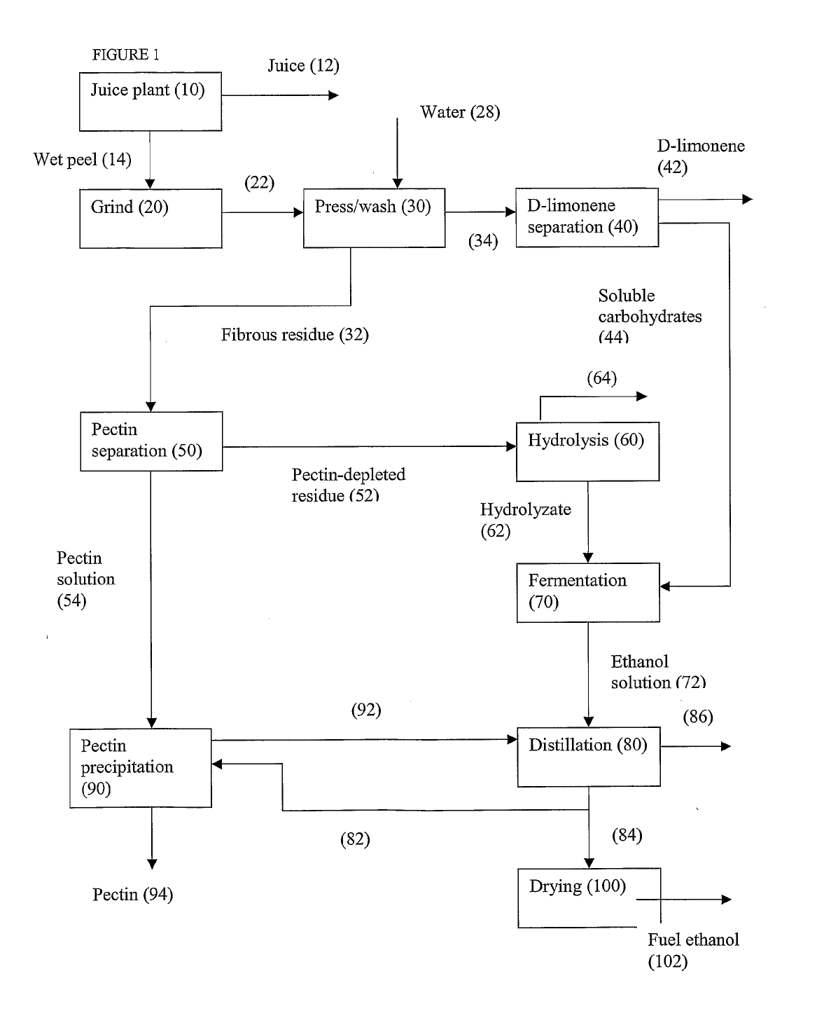

FIG. 1 shows a flow diagram of an exemplary embodiment of a method for processing citrus fruit to separate pectin and to produce an alcohol (e.g. ethanol). A citrus juice plant (10) generates juice (12) and wet peel (14) as the major by-product. According to an embodiment of the method, the wet peel along with other by-products such as rag, core, pulp, membranes, frits and the like is ground or milled in (20), using methods such as hammer milling to form ground wet peel (22). The ground wet peel comprises water-soluble carbohydrates, such as sucrose, glucose and fructose and water-insoluble carbohydrates (polysaccharides), such as cellulose and hemicellulose. It also comprises pectin, flavonoids, organic acids, pigments, flavors, essences, vitamins and D-limonene, according to another embodiment. Soluble carbohydrates are extracted according to another embodiment from the peel (30) to form an aqueous solution of soluble carbohydrates (34) and a fibrous residue (32), which are separated. Extraction may involve pressing of wet peel or pH-adjusted wet peel contacting with possible addition of fresh water at a suitable temperature and various combinations of those.

Stream (34) contains D-limonene according to another embodiment, and can be treated for the separation of the D-limonene for commercial application. The separation (40) may use a method such as distillation, solvent extraction and de-emulsification. According to another embodiment, ethanol or another organic solvent is used for extraction of D-limonene. According to another embodiment, D-limonene is separated by de-emulsification, which may or may not be assisted by the use of adequate chemical agents. Alternatively, D-limonene is left in the aqueous solution (34) and is separated at a latter step, e.g. after fermentation (70).

The fibrous residue (32) is treated, according to another embodiment for separation of pectin (50). Separation of pectin may be accomplished by contacting with an acid solution. Any relatively strong enough acid is suitable. According to another embodiment, separation is done at pH of about 2 and about 80 C during about 2-3 hours. An aqueous medium comprising pectin (54) is formed and separated from the pectin-depleted fibrous residue (52).

The pectin-depleted fibrous residue (52) is hydrolyzed (60), according to another embodiment, to form a hydrolyzate comprising fermentable carbohydrates (62). Hydrolysis is facilitated, according to another embodiment, by using a chemical catalyst, typically an acidic one, and elevated temperature. According to an alternative embodiment, a biological catalyst is used, e.g. an enzyme or a mixture of enzymes having cellulase activity. Optionally, chemical and biological catalysis are combined, e.g. acid hydrolysis followed by enzymatic hydrolysis. According to an embodiment of the process, the hydrolysis of the pectin-depleted residue fully converts it to soluble components. Alternatively, part of it is left as insolubles. According to another embodiment, those insolubles are separated from the hydrolyzate stream (62) and form a stream of insolubles (64). Those insolubles are of commercial use, e.g. as an ingredient in feed and/or for energy generation. According to another embodiment, those insolubles are mixed with wet peel to form citrus pulp pellets (CPP).

According to the exemplary embodiment as shown in FIG. 1, extracted carbohydrates (44) and carbohydrates formed on hydrolysis (62) are fermented (70) to form a fermentation product (72). According to another embodiment, enzymatic hydrolysis of the residue is combined with fermentation, e.g. conducted in the same vessel. Various fermentation products could be formed and more than one fermentation product is generated according to another embodiment. Ethanol is a product of fermenting at least part of the carbohydrates in streams (44) and/or (62), according to another embodiment. The formed ethanol solution (broth) may be purified using known separation techniques, such as distillation (80), forming a concentrated ethanol solution (84). The non-volatile components of the fermentation broth (86) are suitable as feed ingredients and for other applications, such as for energy generation. According to another embodiment, those non-volatile components are mixed with wet peel to form citrus pulp pellets (CPP) of improved value. The concentrated ethanol solution could be dried (100), e.g. on molecular sieve, to form fuel-grade ethanol (102).

According to the exemplary embodiment as shown in FIG. 1, pectin (94) is separated from the aqueous solution containing it (90). According to another embodiment, concentrated ethanol is used to facilitate pectin precipitation. A stream of concentrated ethanol solution (82) may be mixed with the pectin-containing stream (54), whereby pectin precipitates and is separated from the aqueous solution, which contains ethanol (92). That ethanol could be separated from the solution by distillation.

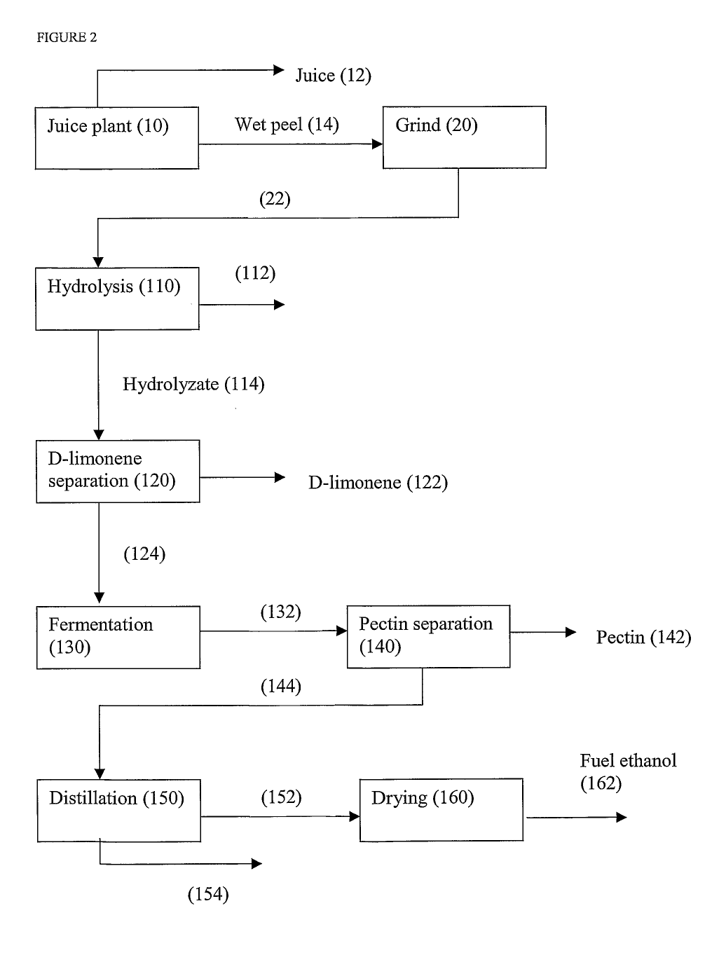

FIG. 2 shows a flow diagram of an alternative embodiment. A citrus juice plant (10) generates juice (12) wet peel and other by-products as described earlier (14). According to an embodiment of the method, the wet peel is ground or milled in (20), using methods such as a hammer mill and forming ground wet peel as the predominant by-product (22). The ground wet peel comprises water-soluble carbohydrates, such as sucrose, glucose and fructose and water-insoluble carbohydrates (polysaccharides), such as cellulose and hemicellulose. It also comprises pectin and D-limonene, according to another embodiment.

The ground wet peel (22) is hydrolyzed (110), according to another embodiment, to form a hydrolyzate comprising fermentable carbohydrates (114). Those fermentable carbohydrates include products of hydrolyzing insoluble carbohydrates and, according to another embodiment, also soluble carbohydrates present initially in the ground, wet peel. Hydrolysis is facilitated, according to another embodiment, by using a chemical catalyst, typically an acidic one, and elevated temperature. According to an alternative embodiment, a biological catalyst is used, e.g. an enzyme or mixture of enzymes having cellulase activity. Optionally, chemical and biological catalysis are combined, e.g. acid hydrolysis followed by enzymatic hydrolysis. The conditions in hydrolysis are chosen, according to another embodiment, so that hydrolysis is selective in the sense that pectin doesn't hydrolyze or so that its hydrolysis is limited. Enzymatic catalysis is suitable for such selective hydrolysis.

According to an embodiment of the process, the hydrolysis of the ground wet peel fully converts it to soluble components. Alternatively, part of it is left as insolubles. According to another embodiment, those insolubles are separated from the hydrolyzate stream (114) and form a stream of insolubles (112). Those insolubles are of commercial use, e.g. as an ingredient in feed and/or for energy generation. According to another embodiment, those insolubles are mixed with wet peel to form citrus pulp pellets (CPP).

The hydrolyzate stream (114) contains D-limonene according to another embodiment, and can be treated for the separation of the D-limonene (122) for commercial application. The separation (120) may use a method such as distillation, solvent extraction and de-emulsification. According to another embodiment, ethanol is used for extraction of D-limonene. Alternatively, D-limonene is left in the hydrolyzate stream (114) and is separated at a latter step, e.g. after fermentation (130).

According to the embodiment as shown in FIG. 2, extracted carbohydrates and carbohydrates formed on hydrolysis in the hydrolyzate stream (114) are fermented to form a fermentation product in a fermentation operation (130). Various fermentation products could be formed and more than one fermentation product is generated according to another embodiment. Ethanol is a product of fermenting at least part of the carbohydrates in stream (114), according to another embodiment. The formed ethanol solution (broth) (144) is purified using known separation techniques, such as distillation (150), forming a concentrated ethanol solution (152). The non-volatile components of the fermentation broth (154) are suitable as feed ingredients and for other applications, such as for energy generation. According to another embodiment, those non-volatile components are mixed with wet peel to form citrus pulp pellets (CPP) of improved value. The concentrated ethanol solution (152) could be dried (160), e.g. on molecular sieve, to form fuel-grade ethanol (162).

The hydrolyzate stream (114) comprises pectin. According to another embodiment pectin is separated from the solution to form a commercial product. According to another embodiment, separation is by pectin precipitation. Such separation can be done at various stages of the process, e.g. prior to D-limonene separation, simultaneously with it, or after it. According to another embodiment, pectin is separated by precipitation and precipitation is facilitated by the presence of ethanol in solution. Ethanol could be added, e.g. via a fraction of the concentrated ethanol solution (152) to the hydrolyzate stream (optionally integrated with D-limonene extraction). Alternatively it is added after D-limonene extraction. According to the embodiment as shown in FIG. 2, pectin is left in the carbohydrate solution fed to the fermentation (124) so that the fermentation broth (132) contains both ethanol and pectin. Pectin (142) may be separated from the broth in (140) by precipitation facilitated by adjusting the water and/or ethanol concentration there. Adjusting may involve concentration through water removal and/or addition of concentrated ethanol solution, e.g. from the distillation step.

The following examples are presented to illustrate the present invention and to assist one of ordinary skill in making and using the same. The examples are not intended in any way to otherwise limit the scope of the invention.

In carrying out the following examples, the following test procedures were used.

### Determination of Pectin Content of Samples

To one gram of dried pectin sample material was added 100 ml of 60% acidified isopropanol. Acidified isopropanol is 60% isopropanol to which concentrated hydrochloric acid has been added at a concentration of 5%. The material in the acidified isopropanol was stirred for 10 minutes, then filtered through a tared filter crucible. The material was washed with six 15 ml washes of the acid isopropanol. The material was then washed with 60% isopropanol, then with azeotrope isopropanol (87% v/v, specific gravity 0.82). The material was dried at 105° C. for 1 hour, then cooled and weighed. Pectin content is the dry weight of the isopropanol-precipitated material over the starting dry weight of the sample material.

### Viscosity of Pectin

A 1% weight/volume solution of pectin in water was prepared. 60-120 minutes after dissolution, the temperature of the solution was adjusted to 25° C. The viscosity of the solution was measured in milli-Pascals using a viscometer with a number 1 spindle and a speed of 60. The viscometer utilized was a Brookfield DV-1 Series Viscometer.

### Determination of Galacturonic Acid (Gal A) Content and Degree of Esterification (DE)

Galacturonic acid is a measure of the purity of the pectin, and DE is a measure of the functionality of the pectin.

In the procedure for determining galacturonic acid, 100 ml of distilled water was added to 0.5 g of material that was dissolved in 2.0 ml of azeotrope isopropanol. Using a phenolphthalein indicator solution, the material was titrated with 0.1 M sodium hydroxide until color change. The titration endpoint was then used to quantify the carboxylic acid groups on the galacturonic acid residues.

To the titrated material, 20 ml of 0.5 M sodium hydroxide was added, mixed, and the solution was allowed to stand for 15 minutes at room temperature. Next, 20.0 ml of 0.5 M hydrochloric acid was added and mixed well with the solution. This material was then titrated with 0.1 M sodium hydroxide to a faint pink end point that gives the uncorrected saponification titer. The titer is corrected by subtracting the titration volume of 100 ml of distilled water that has been mixed first with the 20 ml of 0.5 M sodium hydroxide, and then the 20.0 ml of 0.5 M hydrochloric acid. The DE is the second titer divided by the first and second titers added together, giving a % substitution of the ester.

Analysis of Sucrose, Glucose and Fructose

Samples were analyzed for sugars by first diluting with water if necessary, then filtering the samples with a 0.45 micron nylon Whatman filter. The samples were mixed with an ion exchange resin (1:1 mixture of Dowex 66 and Dowex 88 resins) and shaken vigorously. The samples were analyzed using an isocratic flow through a Biorad HPX-42C calcium column at 85° C. using water as the eluent. The injection size was 4 microliters and the flow rate was 0.9 ml/min and a refractive index detector was used. Dowex 66 and Dowex 88 resins are available from Dow Chemical Company, Midland, Mich.

### EXAMPLES 1-28

In examples 1-28 there are described procedures for obtaining pectin from the peels of oranges. Wet orange peel was treated with nitric acid, in examples 1-8 to release the pectin. Wet orange peel was treated with Genencor International's Multifect A40 enzyme, in examples 9-13 to release the pectin. In example 14, wet orange peel was treated with Novozyme's Celluclast 1.5 L enzyme to release the pectin. In examples 15-20, Genencor International's Multifect A40 enzyme and Biocatalysts Depol 740 L enzyme was used on wet orange peel either alone or in combination to release the pectin. In example 21, no enzyme was added and no pectin recovery was observed. In example 22 Biosun's Cellulase FG enzyme was used to treat wet orange peel to release pectin. In examples 23-26, Genencor International's Multifect A40 enzyme in combination with Biocatalyst's D692 L was added to wet orange peel to release pectin. In examples 27 and 28, Biocatalyst's Depol 740 L enzyme was added to wet orange peel in combinations with either Genencor International's Multifect A40 or Speczyme CP.

In carrying out the examples, the following procedure was practiced.

The size of frozen orange peel chunks was reduced by grinding in a commercial, home size meat grinder (Model #MM6386; Maverick Industries, Inc., Edison N.J. 08837). In order to inactivate pectin methyl esterase enzymes several flasks containing a starting wet weight of either 250 gm or 100 gm of peel each were blanched with 250 to 500 ml of water each by raising the temperature to 80° C. or 100° C. on a hot plate and holding it for different times as indicated in Table 1. Samples 9 through 12 in Table 1 were adjusted to pH 1.7 with nitric acid and were blanched at pH 1.7. The percent moisture of the peel was determined to be 79.1% by drying a portion in an 85° C. oven overnight. The starting 250 or 100 gm material was divided in half after blanching and the starting dry weights used in the experiment are indicated in Table 1.

After blanching, the solids were recovered by filtration through cheesecloth and the wet peel was treated with either nitric acid or enzymes to hydrolyze the cellulosic matrix and release pectin and soluble sugars. The acid hydrolysis of the solids was carried out at 80° C. or 95° C. for 1 or 3 hours in 1 M nitric acid. The enzymatic hydrolyses of the solids were carried out at 50-55° C. for up to 72 hours, using commercially available cellulase preparations (either Genencor A40 or Novozymes Celluclast 1.5 L). After the hydrolysis was stopped, the dry solids were removed by filtration through cheesecloth. In all cases, the amount of dry solids was significantly less than the amount of starting material and, as can be seen in Table 1, the enzyme treatment was more effective than the acid treatment in hydrolyzing the orange peel. This is shown by comparing the starting sample dry weight column with the residual peel dry weight column.

To the hydrolysis filtrate an equal volume of absolute ethanol was added and the resulting precipitated material was filtered on Whatman filter paper, then washed with 3×100 ml of cold ethanol. The final amount recovered, that is the ethanol insoluble pectin fraction, is shown in the column labeled “pectin fraction dry weight” in Table 1. The material was removed from the filter paper, dried and analyzed for pectin. For pectin analyses (1% Viscosity, Galacturonic acid (Gala) and Degree of Esterification (DE)), individual samples were combined as designated in the table below. Pectin is primarily composed of the methylated ester of polygalacturonic acid and determination of galacturonic acid and degree of esterification give an indication of the amount and quality of pectin present in a sample. As shown in Table 1, in all cases the enzyme treatment yielded a higher recovery of pectin than the acid treatment. The ethanol filtrate of selected samples from Table 1 was analyzed for glucose, fructose, and sucrose, and the amount of total sugars based on the starting dry solids (ds) were calculated. These sugars are available for other uses, such as a substrate for fermentation. The sugar analyses are shown in Table 2.

TABLE 1
Pectin Recovery from Enzymatic and Acid Treatment of Orange Peel

| | | | | | | | | | | | | % | | | |
|---|---|---|---|---|---|---|---|---|---|---|---|---|---|---|---|
| | | | | | | | | | | Re- | | Pectin | | | |
| | SAM- | | | | | | | HY- | HY- | sidual | Pectin | Re- | | | |
| SAM- | PLE | BLANCH | | | HY- | | | DROL | DROL | Peel | Fraction | covery | 1% Vis- | | |
| PLE | DRY | TIME | BLANCH | BLANCH | DROL | ENZYME | | TEMP | TIME | DRY | DRY | from | cosity | GalA | DE |
| # | WT (g) | (min) | TEMP C. | pH Adjust | TYPE | AMT (ml) | | (C.) | (Hr) | WT (g) | WT (g) | Start | (mPas) | (%) | (%) |
| 1 | 26.14 | 3 | 100 | No | Acid | 0 | | 80 | 1 | 10.82 | 1.2478 | 4.8 | 33 | 61.0 | 66.9 |
| 2 | 26.14 | 3 | 100 | No | Acid | 0 | | 80 | 1 | 11.98 | 3.0498 | 11.7 | | | |
| 3 | 26.14 | 3 | 100 | No | Acid | 0 | | 80 | 3 | 11.89 | 3.2805 | 12.5 | 16 | 64.6 | 66.7 |
| 4 | 26.14 | 3 | 100 | No | Acid | 0 | | 80 | 3 | 10.04 | 3.13 | 12.00 | | | |
| 5 | 26.14 | 3 | 100 | No | Acid | 0 | | 95 | 3 | 6.78 | 6.4 | 24.50 | 5 | 59.2 | 62.8 |
| 6 | 26.14 | 3 | 100 | No | Acid | 0 | | 95 | 3 | 6.95 | 5.54 | 21.20 | | | |
| 7 | 26.14 | 3 | 100 | No | Acid | 0 | | 95 | 1 | 11.14 | 3.0681 | 11.80 | 16 | 69.5 | 67.5 |
| 8 | 26.14 | 3 | 100 | No | Acid | 0 | | 95 | 1 | 7.81 | 3.84 | 14.70 | | | |
| 9 | 20.91 | 5 | 80 | pH 1.7 | Enzyme | 0.2 | | 55 | 5.45 | 1.95 | 8.02 | 38.40 | 6 | 45.8 | 62.5 |
| | | | | | A40 | | | | | | | | | | |
| 10 | 20.91 | 5 | 80 | pH 1.7 | Enzyme | 0.2 | | 55 | 18 | 1.46 | 7.2 | 34.40 | | | |
| | | | | | A40 | | | | | | | | | | |
| 11 | 20.91 | 5 | 80 | pH 1.7 | Enzyme | 2 | | 55 | 5.45 | 0.37 | 8.07 | 38.60 | 4 | 48.4 | 61.0 |
| | | | | | A40 | | | | | | | | | | |
| 12 | 20.91 | 5 | 80 | pH 1.7 | Enzyme | 2 | | 55 | 18 | 0.29 | 8.24 | 39.40 | | | |
| | | | | | A40 | | | | | | | | | | |
| 13 | 20.91 | 3 | 80 | No | Enzyme | 0.2 | (0 hr), | 50 | 72 | 4.34 | 5.94 | 28.40 | 7 | 47.1 | 61.2 |
| | | | | | A40 | 0.2 | (24 h) | | | | | | | | |
| 14 | 20.91 | 3 | 80 | No | Enzyme | 0.6 | (0 hr) | 50 | 72 | 4.06 | 5.58 | 26.70 | 10 | 49.7 | 64.4 |
| | | | | | Cellu- | 0.6 | (24 hr) | | | | | | | | |
| | | | | | clast | | | | | | | | | | |

TABLE 2
Sugar Analysis of the Ethanol Filtrate
after Pectin Recovery and Washing

| SAMPLE No. | glucose | fructose | sucrose | total sugar |
|---|---|---|---|---|
| from Table 1 | g/kgDS | g/kgDS | g/kgDS | g/kgDS |
| 4 | 15.3 | 29.08 | 56.62 | 101 |
| 5 | 18.73 | 43.19 | 92.09 | 154.01 |
| 6 | 16.34 | 38.51 | 45.28 | 100.12 |
| 7 | 11.33 | 40.55 | 76.73 | 128.61 |
| 12 | 69.3 | 30.73 | 0 | 100.03 |
| 14 | 30.51 | 11.78 | 0 | 42.3 |

In Table 3, Depol 740 L, a ferulic acid esterase preparation from Biocatalyst's (Wales UK) and BioSun Cellulase FG (Tampa Fla.) were added alone or with Genencor's A40. In all cases, pectin recovery was greater than 20%. These enzymes can be used to prepare pectin from orange peel.

TABLE 3
Preparation of Pectin from Orange Peel using Enzyme Treatment.

| | | | | | | | | Residual | Pectin | % Pectin |
|---|---|---|---|---|---|---|---|---|---|---|
| SAMPLE | SAMPLE | BLANCH | BLANCH | Blanch | ENZ. NAME& | HYDROL | HYDROL | Peel | Fraction. | Recovery |
| # | DRY WT (g) | TIME (min) | TEMP C. | pH Adjust | AMT (ml) | TEMP (c.) | TIME (Hr) | DRY WT (g) | DRY WT (g) | from Start |
| 15 | 20.91 | 60 | 100 | No | A40-0.2 | 55 | 18 | 6.54 | 4.86 | 23.2 |
| 16 | 20.91 | 5 | 80 | pH 1.7 | A40-0.2 | 55 | 18 | 0.75 | 8.99 | 43 |
| | | | | | D740L-0.2 | | | | | |
| 17 | 20.91 | 5 | 80 | pH 1.7 | D740L-0.2 | 55 | 4.45 | 0.64 | 6.07 | 29 |
| 18 | 20.91 | 5 | 80 | pH 1.7 | D740L-0.2 | 55 | 14.5 | 0.56 | 4.36 | 20.8 |
| 19 | 20.91 | 5 | 80 | pH 1.7 | D740L-2.0 | 55 | 4.45 | 0.68 | 5.43 | 26 |
| 20 | 20.91 | 5 | 80 | pH 1.7 | D740L-2.0 | 55 | 14.5 | 0.52 | 4.69 | 22.4 |
| 21 | 20.91 | 3 | 80 | No | None | 50 | 72 | 13.45 | 0.01 | 0 |
| 22 | 20.91 | 3 | 80 | No | BioSun | 50 | 72 | 1.18 | 4.72 | 22.6 |
| | | | | | Cellulase FG | | | | | |
| | | | | | 0.6 at 0, 24 h | | | | | |

The experiments summarized in Table 4 involved the use of various mixtures of enzymes. The commercially available enzymes Depol 692 L from Biocatalysts (Wales, UK) which contain ferulic acid esterases, cellulases, and hemicellulases, and Spezyme CP from Genencor (Rochester, N.Y.) which contain cellulases, hemicellulases, and B-glucanases were added in mixtures with Genencor A40 to the peel. High viscosity generated by these enzyme preparations impeded filtration and recovery of pectin-like material.

TABLE 4
Treatment of Orange Peel with Enzyme Mixtures

| | | | | | | | | Residual | Pectin | % Pectin |
|---|---|---|---|---|---|---|---|---|---|---|
| SAMPLE | SAMPLE | BLANCH | BLANCH | Blanch | ENZ. NAME& | HYDROL | HYDROL | Peel | Fraction. | Recovery |
| # | DRY WT (g) | TIME (min) | TEMP C. | pH Adjust | AMT (ml) | TEMP (c.) | TIME (Hr) | DRY WT (g) | DRY WT (g) | from Start |
| 23 | 20.91 | 3 | 100 | No | A40-0.2 | 55 | 3 | 5.69 | 2.46 | 11.7 |
| | | | | | D692L-2 | | | | | |
| 24 | 20.91 | 3 | 100 | No | A40-0.2 | 55 | 18 | 3.05 | None | 0 |
| | | | | | D692L-2 | | | | | |
| 25 | 20.91 | 3 | 100 | No | A40-0.2 | 55 | 18 | 3.82 | None | 0 |
| | | | | | D692L-2 | | | | | |
| 26 | 20.91 | 3 | 100 | No | A40-0.2 | 55 | 18 | 2.72 | 1.17 | 5.6 |
| | | | | | D692L-2 | | | | | |
| 27 | 20.91 | 3 | 100 | No | A40-0.2 | 55 | 18 | 9.4 | 2.51 | 12 |
| | | | | | D740L-0.2 | | | | | |
| 28 | 20.91 | 3 | 100 | No | D740L-0.2 | 55 | 18 | 8.91 | 1.74 | 8.3 |
| | | | | | SpezCP-.05 | | | | | |

### EXAMPLE 29

In this example there is shown the treatment of an orange peel with an enzyme in a 3 liter reactor, to release pectin. The procedure is as follows:

The size of frozen orange peel chunks was reduced by grinding in a commercial, home size meat grinder. In order to inactivate pectin methyl esterase enzymes a starting wet weight of 2464.8 gm of peel was blanched with six liters of water by raising the temperature to 95° C. on a hot plate and holding it at 95° C. for ten minutes. The percent moisture of the peel was determined to be 79.6% by drying a portion in an 85° C. oven overnight. The blanch water was analyzed for sugar content and was found to contain 3.4 g/l sucrose, 5.7 g/l glucose and 6.1 g/l fructose.

After blanching, the solids were recovered by filtration through cheesecloth and the 2100 g wet weight of peel was divided equally into two 3 liter New Brunswick fermenters that each contained 2.4 liters of sterile water. To each fermenter 4.2 ml of Genencor's Multifect A40 enzyme was added. The fermenters were stirred at 500 rpm, maintained at 55° C. and a pH of 5.2 by addition of sodium hydroxide or hydrochloric acid, and incubated for 7 hrs.

At the end of the incubation, the fermenter contents were filtered through cheesecloth and 89.7 g of solids on a dry basis were recovered. This demonstrated that the solids were reduced from the starting 502.8 grams. To the flow-through filtrate an equal volume of absolute ethanol was added and the material was incubated overnight at 4° C. The precipitated material was filtered on Whatman No. 4 filter paper, and washed with 2 volumes of cold ethanol. The material was removed from the filter paper, dried, and analyzed. Solids recovery from the starting material was 21% and this material was determined to be 81.5% pectin by acidified isopropanol. Galacturonic acid residues were determined to be between 46 and 50%, and degree of esterification was determined to be 64.

### EXAMPLE 30

Fermentation of Peel Sugars is shown in this example as follows:

The blanch water from example 29 contained 3.4 g/l sucrose, 5.7 g/l glucose, and 6.1 g/l fructose. Four fermentations were set up. First, inoculum was prepared using a medium containing 0.1% yeast extract and 0.6% glucose at pH 7.0 and 0.2% prilled dry yeast incubated at 30° C. for 20 hours with shaking in a rotary shaker at 150 rpm. A 5% inoculum was added to the four flasks. The controls contained 1% yeast extract and 2% peptone and 1.5% CaCO₃ and either 5 or 50 g/l glucose. The two experimental flasks contained undiluted blanch water to which the 1% yeast extract, 2% peptone, and 1.5% CaCO₃ were added. The first blanch water fermentation had no added sugars except what was already present, and the second had glucose added to 50 g/l. The flasks were incubated for 18 hrs at 30° C. at pH 7.0. The ethanol concentrations are shown in Table 3. The data shows that the sugars in peel blanch water can serve as a substrate for ethanol fermentation, and that no inhibition is observed.

TABLE 5
Ethanol production in Orange Peel Blanch Water

| Sample | Glucose concentration | Ethanol |
|---|---|---|
| Control | 5 g/l | 1.2 g/l |
| Control | 50 g/l | 19.3 g/l |
| Blanch water | 5 g/l | 4.5 g/l |
| Blanch water | 50 g/l | 20.0 g/l |

### EXAMPLES 31-46

In carrying out examples 31-40, the following procedure was practiced.

In order to inactivate pectin methyl esterase enzymes a beaker containing a starting wet weight of 800 gm of coarsely shredded sugar beet was blanched in 2800 mL water by raising the temperature to 95° C. on a hot plate and holding it at 90-95° C. for three minutes. In some examples, the size of the blanched shredded sugar beet pieces was reduced by grinding in a Deluxe Food Grinder (Model #MM6386; Maverick Industries, Inc., Edison, N.J. 08837). The percent moisture of the blanched sugar beet was determined to be 79.1% by drying a portion in a 70° C. oven until a constant weight was obtained. The starting dry weight for each of the examples 31-40 was 20.9 g, as indicated in Table 6.

After blanching, the sugar beet solids were recovered by filtration through cheesecloth. To 100 g aliquots of blanched sugar beet was added either 200 mL of water or 200 mL of 200 mM sodium citrate, pH 4.2. The water-beet suspension was treated with 1 M nitric acid to achieve a pH of 1.6-1.9, while the citrate-beet suspensions were treated with enzymes (either Genencor Multifect A40, Lucigen Comblaster HE-1, Lucigen Comblaster HE-3, Dyadic Acid Cellulase #1 or Dyadic Neutral Cellulase #2) to hydrolyze the cellulosic and/or hemicellulosic matrices and release pectin and soluble sugars. The acid hydrolysis was carried out at 95° C. for 1 hour. The enzymatic hydrolyses were carried out in a shaker-incubator at 50° C. with shaking at 250-270 rpm for 68-70 hours. After the hydrolysis was stopped, the (soluble) dry solids were removed by filtration through cheesecloth. In all cases, the amount of dry solids was significantly less than the amount of starting material. This is shown by comparing the starting sample dry weight column with the residual beet dry weight column.

To the filtrate an equal volume of isopropyl alcohol (IPA) was added, the mixture was stored at 4° C. for up to 72 h, and the resulting precipitated material was filtered on Whatman 1 filter paper, then washed with 3×100 ml of IPA. The final weight of the recovered IPA insoluble pectin is shown in the column labeled “pectin fraction dry weight” in Table 6. The material was dried at 42° C. until a constant weight was obtained.

The results of examples 31-40 are reported in Table 6.

In carrying out examples 41-46, the following procedure was practiced.

Apple pomace was prepared by blending equal amounts of Gala, Braeburn, and Red Delicious apples minus the stems in a Hamilton Beach 14-speed blender (Hamilton Beach/Proctor-Silex, Washington, N.C. 27889) until a thin paste formed. The paste was filtered through cheesecloth to remove the juice. In order to inactivate pectin methyl esterase enzymes a beaker containing a starting wet weight of 800 gm of apple pomace was blanched in 2000 mL water by raising the temperature to 95° C. on a hot plate and holding it at 90-95° C. for three minutes. The percent moisture of the blanched apple pomace was determined to be 88.0% by drying a portion in a 70° C. oven until a constant weight was obtained. The starting dry weight for each of the examples 41-46 was 12.9 g, as indicated in Table 7.

After blanching, the solids were recovered by filtration through cheesecloth. To 100 g aliquots of blanched apple pomace was added either 200 mL of water or 200 mL of 200 mM sodium citrate, pH 4.2. The water-pomace suspension was treated with 1 M nitric acid to achieve a pH of 1.6-1.9 while the citrate-pomace suspensions were treated with enzymes (either Genencor Multifect A40, Lucigen Comblaster HE-1, Lucigen Comblaster HE-3, Dyadic Acid Cellulase #1 or Dyadic Neutral Cellulase #2) to hydrolyze the cellulosic and/or hemicellulosic matrices and release pectin and soluble sugars. The acid hydrolysis was carried out at 95° C. for 1 hour. The enzymatic hydrolyses were carried out in a shaker-incubator at 50° C. with shaking at 250-270 rpm for 70 hours. After the hydrolysis was stopped, the dry solids were removed by filtration through cheesecloth. In all cases except one, the amount of dry solids was significantly less than the amount of starting material. This is shown by comparing the starting sample dry weight column with the residual pomace dry weight column.

To the filtrate an equal volume of isopropyl alcohol (IPA) was added, the mixture was stored at 4° C. for up to 72 h, and the resulting precipitated material was filtered on Whatman 1 filter paper, then washed with 3×100 ml of IPA. The final weight of the recovered IPA insoluble pectin is shown in the column labeled “pectin fraction dry weight” in Table 7. The material was dried at 42° C. until a constant weight was obtained.

The results of examples 41-46 are reported in Table 7.

TABLE 6
Pectin Recovery from Enzymatic and Acid Treatment of Shredded Sugar Beet

| | | SAMPLE | | | | | | | |
|---|---|---|---|---|---|---|---|---|---|
| SAMPLE | | Dry Wt (g) | BLANCH | BLANCH | HYDROLYSIS | ENZYME | VOLUME/WT | | HYDROLYSIS |
| # | Ground | (after blanching) | TIME (min) | TEMP C. | TYPE | NAMES | ENZYME | | LIQUID |
| 31 | no | 20.9 | 3 | 90-95 C. | acid (pH 1.6-1.9) | | | | H2O |
| 32 | no | 20.9 | | | enzyme | A40 | 2 | mL | 200 mM citrate, pH 4.2 |
| 33 | no | 20.9 | | | enzyme | Comblaster HE-1 | 1 | mL | 200 mM citrate, pH 4.2 |
| 34 | no | 20.9 | | | enzyme | Comblaster HE-3 | 1 | mL | 200 mM citrate, pH 4.2 |
| 35 | no | 20.9 | | | enzyme | acid cellulase #1 | 0.03 | g | 200 mM citrate, pH 4.2 |
| 36 | no | 20.9 | | | enzyme | neutral cellulase #2 | 0.05 | g | 200 mM citrate, pH 4.2 |
| 37 | yes | 20.9 | | | acid (pH 1.6-1.9) | | | | H2O |
| 38 | yes | 20.9 | | | enzyme | A40 | 2 | mL | 200 mM citrate, pH 4.2 |
| 39 | yes | 20.9 | | | enzyme | Comblaster HE-3 | 1 | mL | 200 mM citrate, pH 4.2 |
| 40 | yes | 20.9 | | | enzyme | neutral cellulase #2 | 0.05 | g | 200 mM citrate, pH 4.2 |

| | | | | Residual | Pectin | % Pectin |
|---|---|---|---|---|---|---|
| SAMPLE | LIQUID | HYDROL | HYDROL | Beet | Fraction | Recovery |
| # | VOLUME (mL) | TEMP (C.) | TIME (Hr) | Dry Wt (g) | Dry Wt (g) | from Start |
| 31 | 200 | 90-95 C. | 1 | 8.9 | 1.37 | 6.6 |
| 32 | 200 | 50 C. | 70 | 14.6 | 1.48 | 7.1 |
| 33 | 200 | 50 C. | 70 | 15.9 | 0.34 | 1.6 |
| 34 | 200 | 50 C. | 70 | 15.9 | 0.76 | 3.6 |
| 35 | 200 | 50 C. | 70 | 16.4 | 0.77 | 3.7 |
| 36 | 200 | 50 C. | 70 | 16.9 | 0.57 | 2.7 |
| 37 | 200 | 90-95 C. | 1 | 8.1 | 1.34 | 6.4 |
| 38 | 200 | 50 C. | 68 | 14.8 | 0.47 | 2.2 |
| 39 | 200 | 50 C. | 68 | 13.9 | 0.31 | 1.5 |
| 40 | 200 | 50 C. | 68 | 14.2 | 0.46 | 2.2 |

TABLE 7
Pectin Recovery from Enzymatic and Acid Treatment of Apple Pomace

| | | SAMPLE | | | | | | | |
|---|---|---|---|---|---|---|---|---|---|
| SAMPLE | | Dry Wt (g) | BLANCH | BLANCH | HYDROLYSIS | ENZYME | VOLUME/WT | | HYDROLYSIS |
| # | Blended | (after blanching) | TIME(min) | TEMP C. | TYPE | NAMES | ENZYME | | LIQUID |
| 41 | yes | 12.8 | 3 | 90-95 C. | acid (pH 1.6-1.9) | | | | H2O |
| 42 | yes | 12.8 | | | enzyme | A40 | 2 | mL | 200 mM citrate, pH 4.2 |
| 43 | yes | 12.8 | | | enzyme | Comblaster HE-1 | 1 | mL | 200 mM citrate, pH 4.2 |
| 44 | yes | 12.8 | | | enzyme | Comblaster HE-3 | 1 | mL | 200 mM citrate, pH 4.2 |
| 45 | yes | 12.8 | | | enzyme | acid cellulase #1 | 0.03 | g | 200 mM citrate, pH 4.2 |
| 46 | yes | 12.8 | | | enzyme | neutral cellulase #2 | 0.05 | g | 200 mM citrate, pH 4.2 |

| | | | | Residual | Pectin | % Pectin |
|---|---|---|---|---|---|---|
| SAMPLE | LIQUID | HYDROL | HYDROL | POMACE | PECTIN | Recovery |
| # | VOLUME (mL) | TEMP (C.) | TIME (Hr) | Dry Wt (g) | Dry Wt (g) | from Start |
| 41 | 200 | 90-95 C. | 1 | 8.9 | 1.09 | 8.5 |
| 42 | 200 | 50 C. | 70 | 1.5 | 1.75 | 13.7 |
| 43 | 200 | 50 C. | 70 | 12.8 | 0.72 | 5.6 |
| 44 | 200 | 50 C. | 70 | 12.5 | 0.67 | 5.2 |
| 45 | 200 | 50 C. | 70 | 7.6 | 1.22 | 9.5 |
| 46 | 200 | 50 C. | 70 | 7.3 | 0.95 | 7.4 |

From the data in Tables 6 and 7, the following observations may be made:

The data in the columns labeled Residual Beet Dry Weight and Residual Pomace Dry Weight show that both acid treatment and enzymes digest the apple and sugar beet biomass and release pectin that can be precipitated by the addition of isopropyl alcohol. The enzyme that released the most pectin from shredded sugar beet and apple pomace, as measured by the pectin fraction dry weights, is Genencor Multifect A40. The enzymatic hydrolyses of the apple pomace released more pectin than the comparable hydrolyses of the shredded sugar beet (see columns labeled % Pectin Recovery from Start).

However, in all instances, the use of enzyme in the treatment of the sugar beet and the apple pomace was successful in recovering pectin.

The invention has been described with reference to various specific and illustrative embodiments and techniques. However, one skilled in the art will recognize that many variations and modifications may be made while remaining within the spirit and scope of the invention.

1. A method for obtaining pectin from a pectin-containing material comprising:
treating the pectin-containing material in an aqueous medium with enzyme to release pectin from the pectin-containing material thereby forming a pectin solution; and
separating the pectin solution from insoluble residue.

2. The method according to claim 1 wherein the pectin solution is contacted with an alcohol.

3. The method according to claim 1 wherein the pectin-containing material is selected from the group consisting of a citrus fruit, a tropical fruit, an apple, a sugar beet, a sunflower, a soybean, and mixtures thereof.

4. The method according to claim 3 wherein the pectin-containing material is selected from the group consisting of a citrus fruit and an apple.

5. The method according to claim 1 wherein the pectin-containing material is a citrus fruit from which at least a portion of the liquid of the fruit has been removed.

6. The method according to claim 1 wherein the aqueous medium is water.

7. The method according to claim 1 wherein the enzyme is selected from the group consisting of a cellulase enzyme, a hemicellulase enzyme, and a mixture thereof.

8. The method according to claim 1 wherein the weight ratio of aqueous medium to the pectin contained in the pectin-containing material is in a range of about 10 to about 300 parts aqueous medium to about 1 part pectin.

9. The method according to claim 1 wherein the pectin-containing material is ground to a smaller size, prior to or during the treatment with enzyme.

10. The method according to claim 1 wherein the enzyme treatment of the pectin-containing material is carried out under acidic conditions.

11. The method according to claim 10 wherein the enzyme treatment of the pectin-containing material is carried out at a pH of lower than about 5.

12. The method according to claim 11 wherein the pH is lower than about 4.

13. The method according to claim 1 wherein the enzyme treatment of the pectin-containing material is carried out at a temperature of lower than about 100° C.

14. The method according to claim 13 wherein the temperature is lower than about 60° C.

15. The method according to claim 1 wherein the enzyme treatment of the pectin-containing material is carried out for a period of less than about 72 hours.

16. The method according to claim 15 wherein the period is less than about 6 hours.

17. The method according to claim 1 wherein the pectin solution is separated from the insoluble residue by filtration or centrifugation.

18. The method according to claim 2 wherein the alcohol is selected from the group consisting of ethanol and isopropanol.

19. The method according to claim 1 wherein the pectin-containing material is pretreated to stabilize the pectin-containing material by inactivating enzymes in the pectin-containing material.

20. The method according to claim 19 wherein the pretreatment comprises heating the pectin-containing material.

21. The method according to claim 20 wherein the pectin-containing material is heated with hot water at a temperature of about 80° C. to about 100° C., for a period of about 3 to 5 minutes.

22. Pectin produced in accordance with the method of claim 2.

23. Pectin produced in accordance with the method of claim 18.

24. A food composition comprising pectin according to claim 22.

25. A food composition comprising pectin according to claim 23.

26. A beverage composition comprising pectin according to claim 22.

27. A beverage composition comprising pectin according to claim 23.
